(19) United States
(12) Patent Application Publication
(10) Pub. No.: US 2026/0000419 A1
(43) Pub. Date: Jan. 1, 2026

(54) CLOT EXTRACTION SYSTEMS AND METHODS

(71) Applicant: ASAHI INTECC CO., LTD., Seto-shi (JP)

(72) Inventors: Michael LUK, Orange, CA (US); Elijah DE GUZMAN, Irvine, CA (US); Lynn DO, Irvine, CA (US)

(73) Assignee: ASAHI INTECC CO., LTD., Seto-shi (JP)

(21) Appl. No.: 19/251,001

(22) Filed: Jun. 26, 2025

Related U.S. Application Data
(60) Provisional application No. 63/665,435, filed on Jun. 28, 2024.

(51) Int. Cl. A61B 17/221; A61B 17/22

(52) U.S. Cl. CPC A61B 17/221; A61B 2017/22081; A61B 2017/2212

(57) ABSTRACT

A provided clot extraction system can be used for the removal of clot that may have a wall adherent component. The clot extraction system includes a cover sleeve and a telescoping 5-shaft assembly. A coring element having a blade used for physically breaking down clot is coupled to the shaft assembly. A mesh support element is coupled to the shaft assembly. An open end of a mesh is coupled to the mesh support element and a distal end of the mesh is coupled to the shaft assembly. The mesh is used to collect clot within the vessel, such as the clot that the blade of the coring element separates from the vessel wall. The shape of the coring element or the mesh support element may be altered via the shaft assembly.

### CROSS-REFERENCE TO RELATED APPLICATIONS

This application claims the earlier filing date benefit of U.S. Provisional Application No. 63/665,435, filed on Jun. 28, 2024, the entirety of which is incorporated by reference herein.

### TECHNICAL FIELD

The present application relates generally to the medical field. More specifically, the present application relates to clot extraction devices and procedures.

### BACKGROUND

Thrombectomy is a medical procedure for removing blood clots from blood vessels, particularly within the brain or other vital organs, and is commonly employed to treat conditions such as ischemic stroke, deep vein thrombosis (DVT), and pulmonary embolism. The procedure involves the use of specialized devices, often guided by imaging techniques such as fluoroscopy or angiography, to physically extract or dissolve the blood clots. For instance, a delivery catheter may be navigated through the vasculature to reach the site of the blood clot. Specialized devices, such as a stent retriever housed within the delivery catheter and/or an aspiration catheter, are then employed to physically remove or break down the blood clot. Stent retrievers, for example, are designed to trap and remove the clot when deployed within the vessel.

### SUMMARY

The following summarizes some embodiments of the present disclosure to provide a basic understanding of the discussed technology. This summary is not an extensive overview of all contemplated features of the disclosure and is intended neither to identify key or critical elements of all embodiments of the disclosure nor to delineate the scope of any or all embodiments of the disclosure. Its sole purpose is to present some concepts of one or more embodiments of the disclosure in summary form as a prelude to the more detailed description that is presented later.

A new and innovative clot extraction system and device, and related methods, can be used for the removal of clot (e.g., high-volume, long-length mixed clot) that may have a wall adherent component. For example, the clot extraction system may be used for peripheral venous DVT clot removal. In another example, the clot extraction system may be used in the arterial side of a patient's vascular system where there can be stenotic lesions.

The clot extraction system includes a cover sleeve and a telescoping 5-shaft assembly that includes, starting from the innermost shaft, a mesh inner shaft, a mesh middle shaft, a mesh outer shaft, a coring inner shaft, and a coring outer shaft. A coring element having a cutting edge (e.g., blade) used for separating a blood clot from a blood vessel wall, or breaking down a blood clot within a blood vessel, is secured to the coring inner shaft and the coring outer shaft. A mesh support element is secured to the mesh middle shaft and the mesh outer shaft. An open end of a mesh, or mesh element, is coupled to the mesh support element and a closed end of the mesh is coupled to the mesh inner shaft. The mesh is used to collect clot within the vessel, such as the clot that the blade of the coring element separates from the vessel wall or from a larger piece of clot.

The shape of the coring element, such as the angle that the blade forms with an axis of the 5-shaft assembly, is adjustable through movement of the coring inner shaft or the coring outer shaft relative to one another. Similarly, the shape of the mesh support element, such as the angle that arms of the mesh support element coupled to the mesh form with an axis of the 5-shaft assembly, is adjustable through movement of the mesh middle shaft and the mesh outer shaft relative to one another. The length of the mesh can be adjusted through movement of the mesh inner shaft relative to the other shafts.

During operation of the clot extraction system, the coring element may be translated along the axis of the 5-shaft assembly independently from translation of the mesh support element along the axis. In this way, the coring action via the coring element can be independent from the collecting action via the mesh, which may reduce trauma to the vessel and may provide one or more other advantages.

One example of the present systems comprises a first shaft; a second shaft; a third shaft; a fourth shaft; a fifth shaft; a support element-which may comprise a multi-arm support element, such as a support element that is resilient or self-expanding and/or comprises a central region/portion/segment connected (e.g., integrally) to multiple arms, each of which extends from the central segment—coupled to (e.g., in direct contact with each of) the second and third shafts; a mesh having an open end and a second end, wherein the open end of the mesh is coupled to (e.g., in direct contact with) the support element and the second end of the mesh is coupled to (e.g., in direct contact with) the first shaft; and a coring element—which may comprise a multi-arm coring element, such as a coring element that is resilient or self-expanding and/or comprises a central region/portion/segment connected (e.g., integrally) to multiple arms, each of which extends from the central segment—coupled to (e.g., in direct contact with each of) the fourth and fifth shafts. The coring element may include a cutting edge. The open end may be proximally-facing. The second end may be closed, such as with a tip that is a distinct structure coupled to both the mesh and to the first shaft. Each of the first shaft and the tip may be sized to accept a guidewire. No portion of the coring element may be in direct contact with any portion of the mesh. A distal end of the coring element may be positioned proximal of the open end of the mesh and/or proximal of a proximal end of the support element. The cutting edge may comprise multiple portions of multiple arms of the coring element. Another example of the present systems comprises a telescoping shaft assembly including a plurality of shafts; a coring element-which may comprise a multi-arm coring element, such as a coring element that is resilient or self-expanding and/or comprises a central region/portion/segment connected (e.g., integrally) to multiple arms, each of which extends from the central segment—coupled to the telescoping shaft assembly, the coring element including a cutting edge; a support element—which may comprise a multi-arm support element, such as a support element that is resilient or self-expanding and/or comprises a central region/portion/segment connected (e.g., integrally) to multiple arms, each of which extends from the central segment—coupled to the telescoping shaft assembly and spaced apart from the coring element along the telescoping shaft assembly; and a mesh coupled to the support element. The telescoping shaft assembly, the coring element, and the support element may be configured such that the coring element is movable relative to the support element in a direction parallel to an axis extending through the telescoping shaft assembly. The mesh may have an open proximal end and a distal end that is closed, such as with a tip that is a distinct structure coupled to both the mesh and to one of the shafts of the plurality of shafts. Each of the radially smallest of the shafts and the tip may be sized to accept a guidewire. No portion of the coring element may be in direct contact with any portion of the mesh. A distal end of the coring element may be positioned proximal of the open end of the mesh and/or proximal of a proximal end of the support element. The cutting edge may comprise multiple portions of multiple arms of the coring element.

One example of the present apparatuses comprises a plurality of shafts; a mesh comprising a distal end coupled to a first shaft of the plurality of shafts and an open end coupled to a second shaft of the plurality of shafts; and a coring element comprising a first end coupled to a third shaft of the plurality of shafts and a second end coupled to a fourth shaft of the plurality of shafts, the coring element including a cutting edge. The open end of the mesh may face the coring element, and the cutting edge of the coring element may face away from the mesh. The coring element may comprise a multi-arm coring element, such as a coring element that is resilient or self-expanding and/or comprises a central region/portion/segment connected (e.g., integrally) to multiple arms, each of which extends from the central segment, and in which portions of multiple arms form the cutting edge.

One example of the present systems, and more specifically one of the present clot extraction systems, includes a cover sleeve and a telescoping three-shaft system that includes an inner shaft, a middle shaft, and an outer shaft; a coring element, which may comprise a multi-arm coring element as described above, that has a proximal end coupled to a distal end of the outer shaft, and a distal end that is coupled to a distal end of the middle shaft; a support element, which may comprise a multi-arm support element as described above, that has a proximal end and a distal end that are coupled to the inner shaft such that the proximal end of the support element is fixed axially along the inner shaft while the distal end of the support element is slidable along the inner shaft; and a mesh having a proximal end coupled to the proximal end of the support element, and a distal end coupled to the distal end of the inner shaft. In another example, both the proximal and distal ends of the coring element may instead be coupled to the outer shaft such that the proximal end of the coring element is fixed axially along the outer shaft while the distal end of the coring element is slidable along the outer shaft. Further, in such other example, the proximal end of the support element is coupled to the distal end of the outer shaft (rather than to the inner shaft), and the distal end of the support element is coupled to a distal end of the middle shaft (rather than to the inner shaft).

An example of the present clot extraction methods include deploying a clot extraction system within a vascular system of a patient. The deploying may include introducing the clot extraction system into the vascular system through an introducer (e.g., an introducer sheath, such as one that includes a funnel sheath that is open at a distal end thereof and that faces distally) and advancing the clot extraction system (e.g., over a guidewire) to a desired position within the vascular system. A cover (e.g., a cover sleeve) of the system may then be retracted to deploy a coring element, which may comprise a multi-arm coring element as described above, a support element, which may comprise a multi-arm support element as described above, and a mesh of the clot extraction system. The method may include changing a height (e.g., a radial height, meaning a height taken in a direction perpendicular to an axis of the system) of the coring element, the support element, or both. The method may include retracting (withdrawing) the coring element a first amount (or length or distance) through the vascular system while the support element, and thereby the mesh due to the mesh being secured to the support element, remains stationary. Stated differently, the coring element may be retracted relative to the support element and to the mesh. Such retraction may remove at least a portion of a clot from a wall of a vessel of the vascular system via a blade of the coring element. The first amount may be equal to a length of the coring element between a proximal end and a distal end of the coring element. The method may include retracting the support element and the mesh a second amount through the vascular system while the coring element remains stationary. Stated differently, the support element and the mesh may be retracted relative to the coring element. The second amount may equal, or approximately equal, the first amount. Such retraction of the support element and the mesh may collect at least some of the portion of the clot that was removed from the wall of the vessel by the retraction of the coring element. The method may include reducing the radial height of the blade, retracting the coring element again, and retracting the support element and the mesh again; the method may include repeating such retractions as many time(s) as needed as the diameter of the vessel decreases.

Continuing with the present methods, alternatively, the sequence may include setting (or fixing) a radial height of blade, retracting the clot extraction system (as a whole) for the first amount through the vascular system, reducing the radial height of the blade, and retracting the clot extraction system (as a whole) another first amount. In this alternative sequence, the coring element first breaks down clot during the first retraction of the clot extraction system, and then, after the radial height of the blade is reduced, the coring element breaks down additional clot while the mesh collects the previously broken down clot during the second retraction of the clot extraction system.

Continuing with the present methods, in either embodiment of the sequence described above, the coring that results from retracting the coring element (and contacting clot with the blade) may be independent from the collecting that results from clot (whether cored or not) collecting in the mesh that results from retracting the mesh. Benefits of this separation are described below.

In some embodiments of the present methods, the radial height of the blade and the radial height of the open end of the mesh can be controlled separately, and to different sizes, when desired. For example, the coring may be “turned off” when desired, such as by retracting the coring element within the cover sleeve when not needed, such as while the mesh, when containing or full of clot, is retracted (e.g., pulled through and) out of the patient's vascular system, which eliminates a force of the blade against the vessel wall. In some embodiments of the present methods, the coring element may be retracted within the cover sleeve after each amount the coring element is retracted, while the mesh collects the clot material (separated or otherwise), and the coring element is deployed again (e.g., once such collecting is complete to the satisfaction of the operator) to core an additional amount of the vascular system.

The term “coupled” is defined as joined together, although not necessarily directly, and not necessarily mechanically; two items that are “coupled” may be unitary with each other. The terms “a” and “an” are defined as one or more unless this disclosure explicitly requires otherwise. The term “end” refers to a concluding portion of the referenced structure and is not limited to the terminus of the referenced structure. Herein, translational movement, unless context indicates otherwise, is movement proximal or distal to the operator. The terms “substantially” and “approximately” are each defined as largely but not necessarily wholly what is specified—and include what is specified—as understood by a person of ordinary skill in the art. In any disclosed embodiment, the terms “substantially” and “approximately” may each be substituted with “within [a percentage] of” what is specified, where the percentage includes 0.1, 1, 5, and 10 percent.

Furthermore, all numerical ranges herein should be understood to include all integers, whole or fractions, within the range, inclusive of the ends of the ranges. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The terms “comprise” and any form thereof such as “comprises” and “comprising,” “have” and any form thereof such as “has” and “having,” and “include” and any form thereof such as “includes” and “including” are open-ended linking verbs. As a result, an apparatus or system that “comprises,” “has,” or “includes” one or more elements possesses those one or more elements but is not limited to possessing only those elements. Likewise, a method that “comprises,” “has,” or “includes” one or more steps possesses those one or more steps but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above in order to change the scope of a given claim from what it would otherwise be using the open-ended linking verb.

An apparatus or system that is configured in a certain way is configured in at least that way, but it can also be configured in other ways than those specifically described. Furthermore, reference in the appended claims to an apparatus or system or a component of an apparatus or system being adapted to or configured to perform a particular function encompasses that apparatus, system, or component, whether or not it or that particular function is activated, turned on, or unlocked, as long as that apparatus, system, or component is so adapted or configured.

The feature or features of one embodiment may be applied to other embodiments, even though not described or illustrated, unless expressly prohibited by this disclosure or the nature of the embodiments.

Some details associated with the embodiments are described above and others are described below.

Additional features and advantages of the disclosed methods and apparatuses are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

The following drawings illustrate by way of example and not limitation. For the sake of brevity and clarity, every feature of a given structure is not always labeled in every figure in which that structure appears. Similar components or features may have the same reference label. Further, various components of the same type may be distinguished by following the reference label with a second label that distinguishes among the similar components. Identical reference numbers do not necessarily indicate an identical structure. Rather, the same reference number may be used to indicate a similar feature or a feature with similar functionality, as may non-identical reference numbers.

FIG. 1 is a box diagram of an example clot extraction system, according to an aspect of the present disclosure.

FIG. 2 is a perspective view of the clot extraction system, according to an aspect of the present disclosure.

FIG. 3 is a side view of the clot extraction system showing an outline of a mesh, according to an aspect of the present disclosure.

FIG. 4 is a side view of a shaft assembly of the clot extraction system, according to an aspect of the present disclosure.

FIG. 5A is a perspective view of a first embodiment of a coring element, according to an aspect of the present disclosure.

FIG. 5B is a perspective view of a second embodiment of a coring element, according to an aspect of the present disclosure.

FIG. 6 is a perspective view of a collection mesh, according to an aspect of the present disclosure.

FIG. 7 is an exploded view of the collection mesh, according to an aspect of the present disclosure.

FIG. 8A is a perspective view of a first embodiment of the support element of the collection mesh, according to an aspect of the present disclosure.

FIG. 8B is a perspective view of a second embodiment of the support element of the collection mesh, according to an aspect of the present disclosure.

FIG. 9 is a perspective view of a proximal end of a mesh, according to an aspect of the present disclosure.

FIG. 10 is a perspective view of the proximal end of the mesh attached to the support element, according to an aspect of the present disclosure.

FIG. 11 is a side view of the coring element, according to an aspect of the present disclosure.

FIG. 12A to 12C are a series of side views of the coring element coupled to the shaft assembly, according to an aspect of the present disclosure.

FIG. 13 is a perspective view of a distal end of the coring element relative to a cap for coupling the distal end to a shaft of the shaft assembly, according to an aspect of the present disclosure.

FIG. 14 is a top view of the coring element coupled to shafts of the shaft assembly, according to an aspect of the present disclosure.

FIG. 15 is a side view of the mesh having a length distal of the open end that has substantially the same shape perpendicular to an axis centered within the mesh, according to an aspect of the present disclosure.

FIG. 16 is a side view of the mesh having a shape that tapers toward the distal end of the mesh, according to an aspect of the present disclosure.

FIG. 17 is a side view of the mesh having a bump, according to an aspect of the present disclosure.

FIG. 18 is a side view of the mesh having multiple bumps, according to an aspect of the present disclosure.

FIGS. 19A to 19C are a series of top views of the clot extraction system with different positionings of a cap of the clot extraction system showing compression of the mesh, according to an aspect of the present disclosure.

FIGS. 20A to 20C are a series of side views demonstrating a method of extracting clot with the clot extraction system, according to an aspect of the present disclosure.

### DETAILED DESCRIPTION

The detailed description set forth below, in connection with the appended drawings, is intended as a description of various configurations and is not intended to limit the scope of the disclosure. Rather, the detailed description includes specific details for the purpose of providing a thorough understanding of the inventive subject matter. It will be apparent to those skilled in the art that these specific details are not required in every case and that, in some instances, well-known structures and components are shown in block diagram form for clarity of presentation.

A new and innovative clot extraction system and device, and methods for their use, can be used for the removal of clot (e.g., high-volume, long-length mixed clot) that may have a wall adherent component. For example, the clot extraction system may be used for peripheral venous DVT clot removal. In another example, the clot extraction system may be used for the arterial side of a patient's vascular system where there can be plaque, clot, or stenotic lesion that needs to be extracted. The clot extraction system includes a cover sleeve and a telescoping 5-shaft assembly that includes, starting from the innermost shaft, a mesh inner shaft, a mesh middle shaft, a mesh outer shaft, a coring inner shaft, and a coring outer shaft. A coring element having a blade or cutting edge used for physically separating clot from a vessel wall is coupled to the coring inner shaft at one end of the coring element and to the coring outer shaft at the end of the coring element including the blade.

A mesh support element is coupled to the mesh middle shaft at one end of the mesh support element and to the mesh outer shaft at the other end of the mesh support element. An open, proximally-facing end of a mesh is coupled to the other end of the mesh support element and a second, distal end of the mesh is closed, such as by a distal tip coupled to the mesh inner shaft. The mesh is used to collect clot within the vessel, such as the clot that the blade of the coring element separates from the vessel wall. Here, “open” in “open end” means that this end is the end of mesh into which clot material passes to enter the mesh.

The shape of the coring element, such as the angle that the blade forms with an axis of the 5-shaft assembly, is adjustable through movement of the coring inner shaft and/or the coring outer shaft relative to one another. Similarly, the shape of the mesh support element, such as the angle that arms of the mesh support element coupled to the mesh form with an axis of the 5-shaft assembly, is adjustable through movement of the mesh middle shaft and/or the mesh outer shaft relative to one another. The length of the mesh can be adjusted through movement of the mesh inner shaft relative to one or more of the other shafts.

During operation of the clot extraction system, the coring element may be translated along the axis of the 5-shaft assembly independently from translation of the mesh support element along the axis. In this way, the coring action via the coring element can be independent from the collecting action via the mesh. Separating these two actions can have various benefits in the clot extraction process. For example, coring a first length of the vessel, followed by collecting the separated clot material along that first length of the vessel, enables controlling a height of the blade and a height of the open end of the mesh based on a diameter of the vessel along the first length. The diameter of the vessel along the first length can be consistent, which reduces the force that the coring element and the support element apply to the vessel wall, since the coring element and the support element are not forcing the vessel to expand along this first length. The heights of the blade and the open end can then be reduced before repeating the coring and subsequent collecting on a second length of the vessel, which has a smaller diameter than the first length.

In another example, the height of the blade and the height of the open mouth of the mesh can be controlled to different sizes when desired. For example, the coring action may be “turned off” when desired. For example, the coring element may be retracted within the cover sleeve when not needed, such as while the mesh full of clot is pulled through and out of the patient's vascular system, which eliminates the force of the blade against the vessel wall when coring is not needed. In another example, cutting the clot that is adhering to a vessel wall from the vessel wall in front of the mesh, which pushes the clot material toward the vessel's center, can make it easier to collect the clot material in the mesh in some instances. Additional advantages of the clot extraction system will become apparent with the following description of the figures.

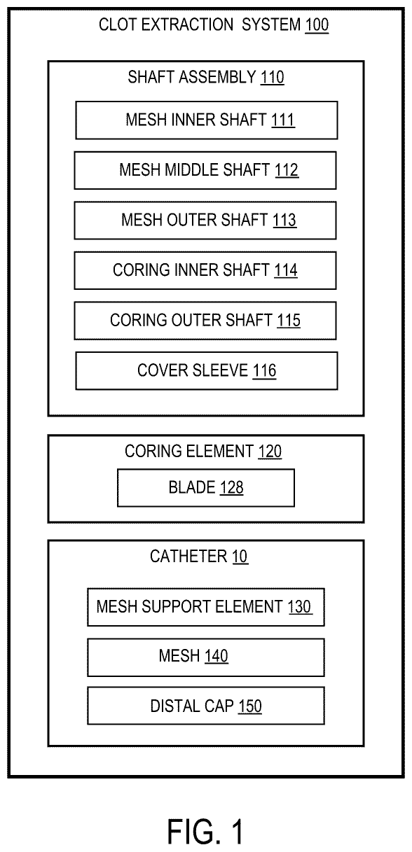

FIG. 1 is a box diagram of an example clot extraction system 100. The clot extraction system 100 includes a shaft assembly 110 coupled to a coring element 120 and to a catheter 10. The catheter 10 includes a mesh support element 130 and a distal cap 150 that each couple a mesh 140 (e.g., a mesh element or clot collecting element) to the shaft assembly 110. In at least some embodiments, the shaft assembly includes a cover sleeve 116 and a 5-shaft system that includes a mesh inner shaft 111 (e.g., a first shaft), a mesh middle shaft 112 (e.g., a second shaft), a mesh outer shaft 113 (e.g., a third shaft), a coring inner shaft 114 (e.g., a fourth shaft), and a coring outer shaft 115 (e.g., a fifth shaft) that are concentrically disposed within one another in a telescoping manner. Each of the sleeve and these shafts may comprise and be referred to as a tube. Specifically, the cover sleeve 116 is disposed around a portion of the coring outer shaft 115, which is disposed around a portion of the coring inner shaft 114, which is disposed around a portion of the mesh outer shaft 113, which is disposed around a portion of the mesh middle shaft 112, which is disposed around a portion of the mesh inner shaft 111.

The relative positions between the mesh inner shaft 111, the mesh middle shaft 112, the mesh outer shaft 113, the coring inner shaft 114, the coring outer shaft 115, and the cover sleeve 116 are adjustable, which allows for the coring element 120 and the catheter 10 to be adjustable. For example, blood vessels within the vasculature of a patient have different sizes and the adjustability of the coring element 120 and the catheter 10 enables the coring element 120 and the catheter 10 to conform to a blood vessel as the blood vessel narrows or widens. In some aspects, a physician may operate a control handle coupled to the clot extraction system 100 to manually manipulate the relative positions between the shafts 111-116 or to allow the coring element 120 and/or catheter 10 to manipulate the relative positions between the shafts 111-116.

The coring element 120 includes a cutting edge (e.g., blade 128) configured to physically separate a blood clot from a vessel wall or break down a blood clot within a vessel. For instance, the coring element 120 may cut both clot material from the vessel's lumen (e.g., its center) and clot material that is adhered to the vessel's wall. The catheter 10 includes the mesh 140 coupled to the mesh support element 130 and to the distal cap 150. A distal end of the mesh 140 is closed by the distal cap 150 coupling the distal end to the shaft assembly 110, whereas a proximal end of the mesh 140 that is coupled to the mesh support element 130 and includes an open region or mouth. Clot within the vessel can thereby be collected into the mesh 140 through the open end, such as the clot removed from a vessel wall by the blade 128.

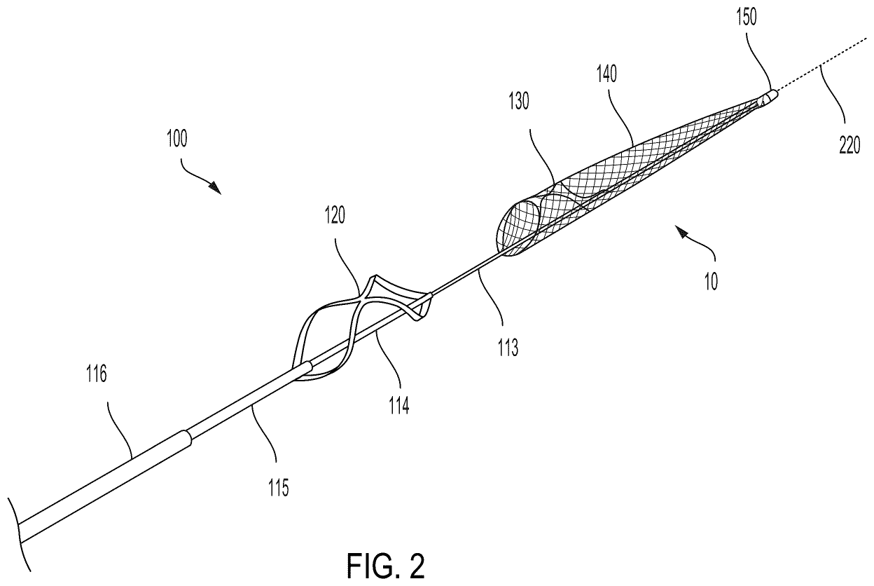

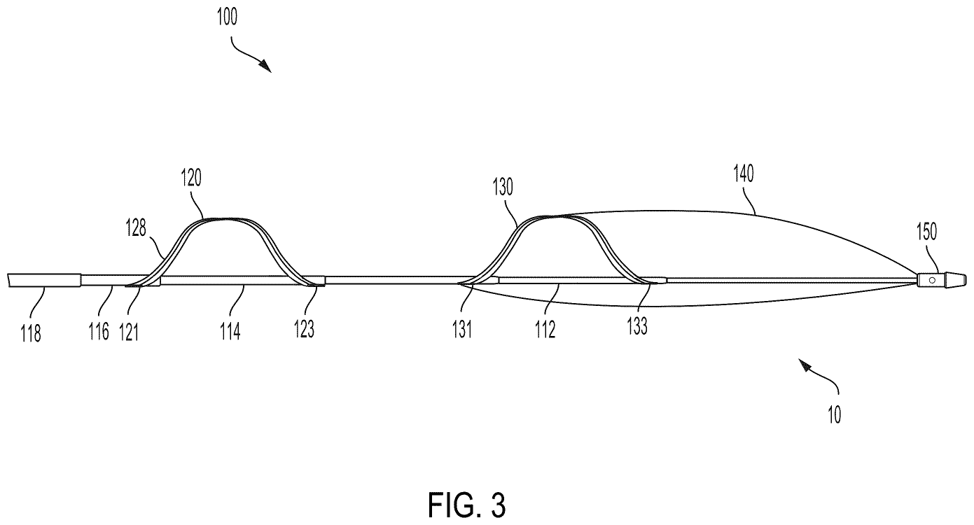

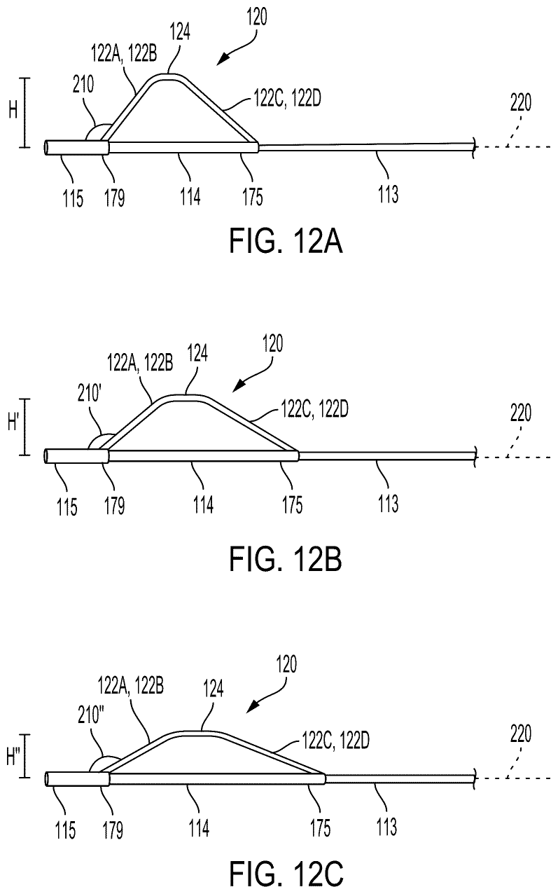

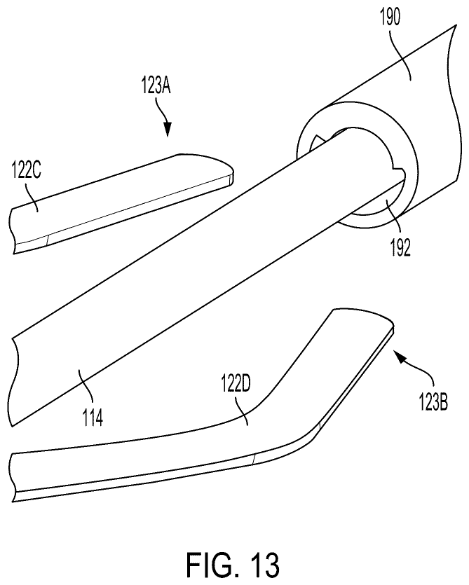

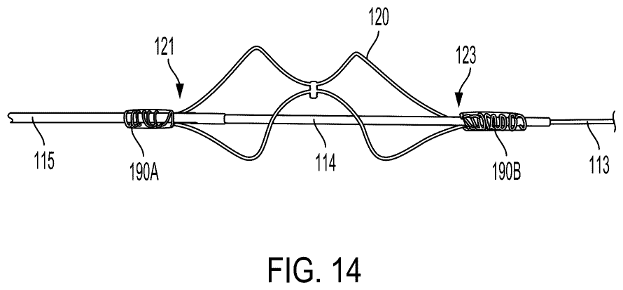

FIGS. 2 to 7 show an example implementation of the clot extraction system 100 and reference is made to each of these figures throughout the description of each of these figures. Referring to FIGS. 2 and 3, a proximal end 121 of the coring element 120 is coupled to the distal end 179 of the coring outer shaft 115. A distal end 123 of the coring element 120 is coupled to the distal end 175 of the coring inner shaft 114. In this way, translation of the coring inner shaft 114 and/or the coring outer shaft 115 relative to one another along an axis 220 of the shaft assembly 110 opens or closes (or reduces or increases the axial length of) the coring element 120 by increasing or decreasing its height (see FIGS. 12A-12C). The axis 220 extends through a center of each of the mesh inner shaft 111, the mesh middle shaft 112, the mesh outer shaft 113, the coring inner shaft 114, the coring outer shaft 115, and the cover sleeve 116. Examples of how the coring element 120 can be coupled to the coring outer shaft 115 and the coring inner shaft 114 are described below in connection with FIGS. 13 and 14.

A proximal end 131 of the mesh support element 130 is coupled to the distal end 171 of the mesh outer shaft 113. A distal end 133 of the mesh support element 130 is coupled to the distal end 167 of the mesh middle shaft 112. A proximal end 141 of the mesh 140, which includes mouth 142 of the mesh 140, is coupled to a proximal end 131 of the mesh support element 130. In this way, translation of the mesh middle shaft 112 or the mesh outer shaft 113 shaft relative to one another along the axis 220 of the shaft assembly 110 opens or closes (or reduces or increases the axial length of) the mesh support element 130 and thereby the mouth 142 of the mesh 140. Examples of how the mesh support element 130 can be coupled to the mesh outer shaft 113 and the mesh middle shaft 112 are described below in connection with FIGS. 13 and 14. The distal cap 150 closes the distal end 143 of the mesh 140 and is coupled to the distal end 163 of the mesh inner shaft 111, thereby also coupling the distal end 143 of the mesh 140 to the distal end 163 of the mesh inner shaft 111.

In some embodiments, the clot extraction system 100 may comprise the distal end of a clot extraction device. For example, the clot extraction system 100 may extend from a control handle of the clot extraction device that is used to control translation of the mesh inner shaft 111, the mesh middle shaft 112, the mesh outer shaft 113, the coring inner shaft 114, the coring outer shaft 115 along the axis 220.

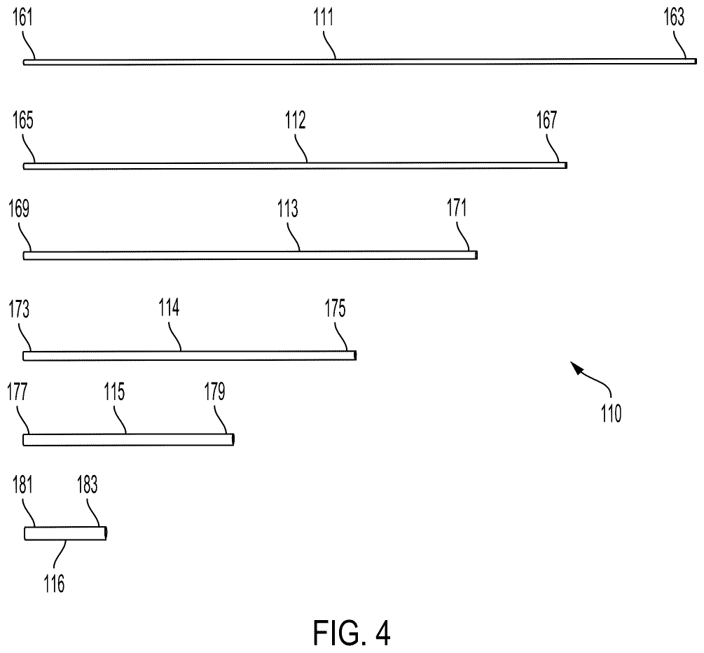

FIG. 4 is an exploded view of an example implementation of the shaft assembly 110. The mesh inner shaft 111 includes a distal end 163 and a proximal end 161, the mesh middle shaft 112 includes a distal end 167 and a proximal end 165, the mesh outer shaft 113 includes a distal end 171 and a proximal end 169, the coring inner shaft 114 includes a distal end 175 and a proximal end 173, the coring outer shaft 115 includes a distal end 179 and a proximal end 177, and the cover sleeve 116 includes a distal end 183 and a proximal end 181. It will be appreciated that the mesh inner shaft 111, the mesh middle shaft 112, the mesh outer shaft 113, the coring inner shaft 114, the coring outer shaft 115, and the cover sleeve 116 are not drawn to scale in FIG. 4.

Each of the mesh inner shaft 111, the mesh middle shaft 112, the mesh outer shaft 113, the coring inner shaft 114, the coring outer shaft 115, and the cover sleeve 116 has a suitable length for a thrombectomy procedure. For instance, the working length of the shaft assembly 110 is capable of reaching all the way to the inferior vena cava (IVC) bifurcation or iliac of the patient's anatomy in order to remove clot from the iliac all the way to the popliteal vein behind the patient's knee. In an example, the mesh inner shaft 111 may have a length within a range of 155 cm to 160 cm. The mesh middle shaft 112 may have a length within a range of 130 cm to 135 cm. The mesh outer shaft 113 may have a length within a range of 115 cm to 120 cm. The coring inner shaft 114 may have a length within a range of 95 cm to 100 cm. The coring outer shaft 115 may have a length within a range of 80 cm to 85 cm. The cover sleeve 116 may have a length within a range of 50 cm to 70 cm. In an example, a wall thickness of each of the mesh inner shaft 111, the mesh middle shaft 112, the mesh outer shaft 113, the coring inner shaft 114, the coring outer shaft 115, and the cover sleeve 116 is within a range of 0.010 cm to 0.020 cm.

In some embodiments, each of the mesh inner shaft 111, the mesh middle shaft 112, the mesh outer shaft 113, the coring inner shaft 114, and the coring outer shaft 115 is constructed of a material that includes a polymer, such as a polyimide. The polymer may be reinforced with a coil or braid to improve kink resistance and allow for greater directional control. The coil or braid may include a metal, such as steel, and more particularly such as stainless steel. In some embodiments, each of the mesh middle shaft 112, the mesh outer shaft 113, the coring inner shaft 114, and the coring outer shaft 115 may be cut out of a hypo tube so as to be a slotted hypo tube, which may be constructed of a metal (e.g., stainless steel or Nitinol). The metal, slotted hypo tube allows for a thin wall, flexibility, and pushability.

In various embodiments, each of the mesh inner shaft 111, the mesh middle shaft 112, the mesh outer shaft 113, the coring inner shaft 114, and the coring outer shaft 115 may include an inner liner configured to provide a low friction surface for another of the shafts or a guidewire to pass through. For example, the inner liner may include a polymer, such as an elastomer, and more particularly such as a thermoplastic elastomer. For example, the inner liner may include polytetrafluoroethylene (PTFE), polyether block amide (PEBA) (e.g., PEBAX®), or PEBA with a slip additive (e.g., EverGlide®).

The cover sleeve 116 keeps (or at least helps to keep) the coring element 120, mesh support element 130, and mesh 140 collapsed during insertion of the clot extraction system 100 in a patient's vasculature, including so that the clot extraction system 100 can reach the iliac or IVC bifurcation without disturbing a clot that is in the illiofemoral popliteal section. The cover sleeve 116 is constructed with multiple layers similar to a typical guide catheter. For example, an outer jacket layer of the cover sleeve 116 may include a polymer, such as an elastomer, and more particularly such as a thermoplastic elastomer. For example, the polymer may include polyether block amide (PEBA) (e.g., PEBAX®), PEBA with a slip additive (e.g., EverGlide®), or Nylon 12. A reinforcing braid or coil layer may include a metal, such as steel, and more particularly such as stainless steel. An inner liner layer may include a polymer, such as an elastomer, and more particularly such as a thermoplastic elastomer. For example, the inner liner may include polytetrafluoroethylene (PTFE), polyether block amide (PEBA) (e.g., PEBAX®), or PEBA with a slip additive (e.g., EverGlide®). In some embodiments, a distal end of the cover sleeve 116 may include a hydrophilic coating.

In some embodiments, a proximal end of the cover sleeve 116 may include one or more depth markers. For example, the depth markers may be imprinted on the surface of the cover sleeve 116. In an example, the depth markers may indicate 5 cm intervals. The depth marks can be used by a physician to determine how far the physician has retracted the clot extraction system 100.

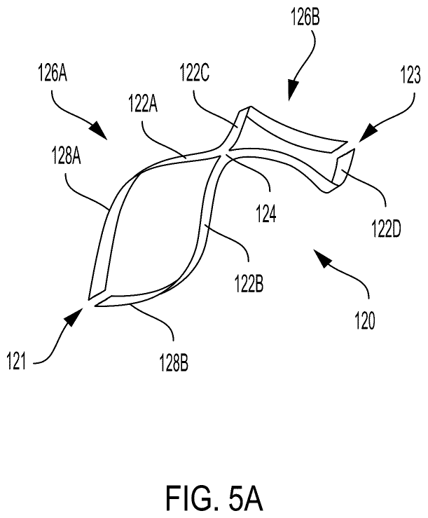

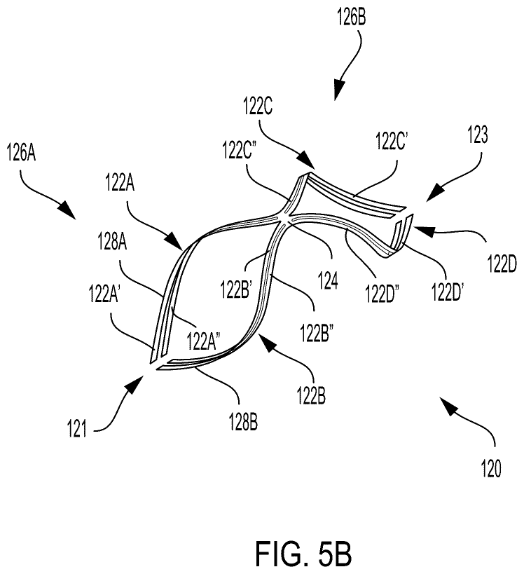

FIG. 5A shows the coring element 120 in isolation. The coring element 120 includes a first portion 126A, which is an example of a proximal portion of the coring element 120 and includes a proximal end 121, and a second portion 126B, which is an example of a distal portion of the coring element 120 and includes a distal end 123. The first portion 126A may be joined to (e.g., integrally formed with) the second portion 126B by a bridge 124, which is an example of a central segment, region, or portion to which the arms of the first and second portions 126A, 126B are connected and from which those arms extend. In some embodiments, the shape of the first portion 126A may be a mirror image of the shape of the second portion 126B relative to the bridge 124.

The first portion 126A includes an arm 122A and an arm 122B. Each of the arms 122A, 122B includes a portion of the blade 128 (or cutting edge) that assists in breaking down blood clots and/or separating adhered blood clots from a vessel wall. For instance, the arm 122A includes a blade (or cutting edge) portion 128A and the arm 122B includes a blade (or cutting edge) portion 128B. The second portion 126B of the coring element 120 includes an arm 122C and an arm 122D. The second portion 126B of the coring element 120 provides support to the first portion 126A so as to support coring action. The second portion 126B is also utilized in adjusting a shape of the coring element 120 as will be described below.

Each of the arms 122A to 122D is formed in at least partially-twisted ribbon-like shape. For instance, near the bridge 124, a first side of the arm 122A faces out-of-the-page whereas that first side is facing into-the-page at the end of the arm 122A away from the bridge 124. In at least some embodiments, the coring element 120 may be cut (e.g., laser cut) out of a tube (e.g., a hypo tube) or a sheet.

In various embodiments, the coring element 120 may include nitinol or another material (e.g., another shape-memory material) suitable for the utility described herein of the coring element 120. In at least some embodiments, the coring element 120 may be heat treated. Each of the arms 122A to 122D may have a cross-sectional thickness within a range of 0.012 to 0.018 inches (0.030 to 0.046 cm) and/or a width within a range of 0.040 to 0.080 inches (0.101 to 0.204 cm). Each of the arms 122A to 122D may be a wire, suture, or other suitable securing member. The thickness and width of each of the arms 122A to 122D may all be the same, or some of the arms 122A to 122D may have different thicknesses and/or widths than other arms 122A to 122D. The blade portions 128A, 128B of the arms 122A, 122B may have a thickness within a range of 0.008 to 0.012 inches (0.020 to 0.031 cm). In other words, the blade (or cutting edge) portions 128A, 128B of the arms 122A, 122B may have a thickness that is less than a thickness of another portion (e.g., the rest of) the arms 122A, 122B. In at least some embodiments, each arm has a width that is greater than its thickness, including, in the depicted embodiment of the coring element 120, over the entire length of the arm except for a very short portion nearest the terminus of each arm (e.g., 90%-99% or more of the length of each arm).

In some embodiments, one or more of the arms 122A, 122B, 122C, or 122D may include two or more arms rather than a single arm as illustrated. For example, FIG. 5B shows an embodiment of the coring element 120 in which the arm 122A includes two arms 122A′ and 122A″, the arm 122B includes two arms 122B′ and 122B″, the arm 122C includes two arms 122C′ and 122C″, and the arm 122D includes two arms 122D′ and 122D″. In this example, only the arms 122A′ and 122B′ include the blade portions 128A and 128B.

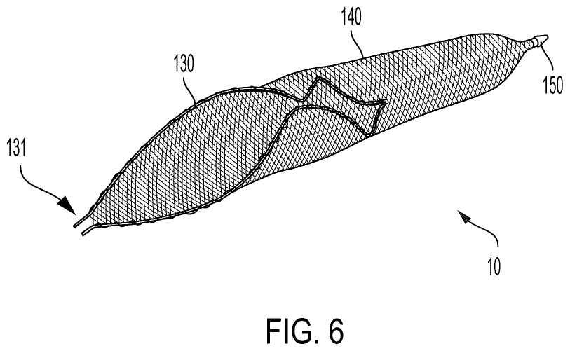

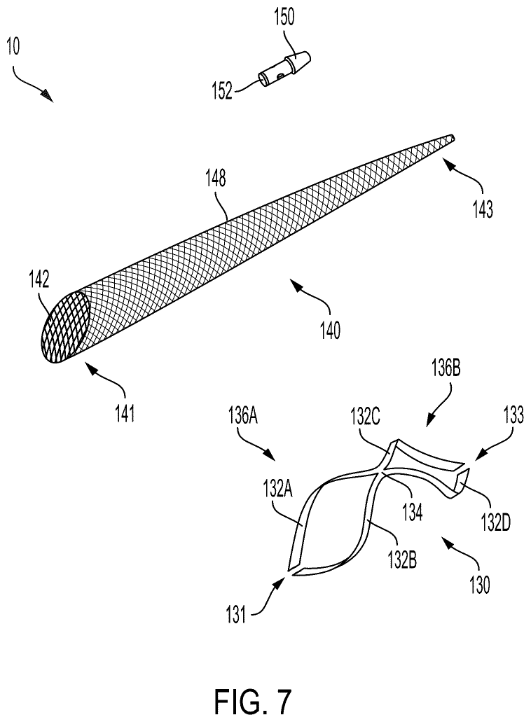

FIGS. 6 and 7 show the catheter 10 in isolation. The mesh 140 includes a proximal end 141 and a distal end 143. The proximal end of the mesh 140 is where clot material enters the mesh 140 and may therefore be characterized as the mouth of the mesh 140. The distal end 143 is closed, such as by the distal cap 150. The distal cap 150 includes a channel 152 extending therethrough.

The mesh 140 may include a plurality of wires braided together. The wires may include nitinol, another suitable shape-memory material, or another suitable material. In the example embodiments described herein, the mesh 140 is treated as including a shape-memory material. In some embodiments, at least some of the wires have a diameter within a range of 0.010 cm to 0.016 cm. In one example, the mesh 140 may be formed using a 48-carrier braider with a full load (i.e., 48 wires) configuration, though the mesh 140 may be formed in other suitable manners. The mesh 140 may be made of single or multiple picks per inch (PPI) segments. In various embodiments, the mesh 140, when the support element is tallest in a radial direction or the mesh is otherwise axially unconstrained, may have a length between the proximal end 141 and the distal end 143 within a range of 5 cm to 19 cm, though other lengths and ranges of lengths are possible. When collapsed (axially constrained), the mesh 140 may have a length that extends up to, for example, 24 cm. The length of the mesh 140 may be selected based on the procedure for which it is to be used. For example, a longer mesh 140 (e.g., 14 cm to 19 cm) may be selected if a large amount of clot is to be collected and pulled out of the patient's vascular system. In another example, a shorter mesh 140 (e.g., 5 cm to 10 cm) may be selected if there is not a large amount of clot to be collected and instead embolic protection (e.g., blocking a path to the superior vena cava or to the right heart and pulmonary system) is needed.

In some embodiments, at least one of the wires of the mesh 140 includes a radiopaque material, such as platinum, which facilitates the position of the mesh 140 being visible during fluoroscopy. In other embodiments, the radiopaque material may be omitted from the mesh 140. In some embodiments, mesh 140 can be made with an elastomer polyurethane cover on the distal end 143 (or an even greater distal portion) of the mesh 140 to prevent embolic loss.

In some embodiments, the proximal end 141 and the distal end 143 of the mesh 140 each have a smaller pore size than a middle portion 148 of the mesh 140. For example, the pore size of the proximal end 141 and distal end 143 may be greater than 0 and less than or equal to 2 millimeters (mm), whereas the pore size of the middle portion 148 may be greater than 2 mm and less than or equal to 5 mm. The larger pore size in the middle portion 148 of the mesh 140 may facilitate excess clots being broken up and squeezed through the pores to exit the mesh 140 if the mesh 140 becomes too full. The average pore size of the proximal end 141 may be similar to the distal end 143 or may be different.

The mesh support element 130 may be structured similarly to the coring element 120, except that the mesh support element 130 does not include a blade. For instance, the mesh support element includes a first portion 136A, which includes a proximal end 131, and a second portion 136B, which includes a distal end 133. The first portion 136A may be joined to the second portion 136B by a bridge 134. In some embodiments, the shape of the first portion 136A may be a mirror image of the shape of the second portion 136B relative to the bridge 134.

The first portion 136A includes an arm 132A and an arm 132B, and the second portion 136B of the mesh support element 130 includes an arm 132C and an arm 132D. The second portion 136B of the mesh support element 130 provides support to the first portion 136A so as to support maintaining the mouth 142 of the mesh 140 open. The second portion 136B is also utilized in the adjustment of the mouth 142 as will be described below.

Each of the arms 132A to 132D is formed in a partially-twisted ribbon-like shape. For instance, near the bridge 134, a first side of the arm 132A faces out-of-the-page whereas that first side is facing into-the-page at the end of the arm 132A away from the bridge 134. In various embodiments, the mesh support element 130 may be cut (e.g., laser cut) out of a tube (e.g., a hypo tube) or a sheet.

In various embodiments, the mesh support element 130 may include nitinol or another material (e.g., another shape-memory material) suitable for the utility described herein of the mesh support element 130. Each of the arms 132A to 132D may have a cross-sectional thickness within a range of 0.012 to 0.018 inches (0.030 to 0.046 cm) and a width within a range of 0.040 to 0.080 inches (0.101 to 0.204 cm). The thickness and width of each of the arms 132A to 132D may all be the same, or some of the arms 132A to 132D may have different thicknesses and/or widths than other arms 132A to 132D. In at least some embodiments, the mesh support element 130 may be heat treated.

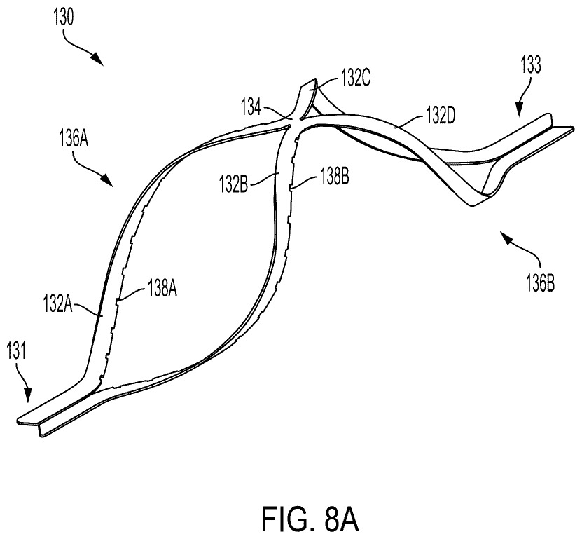

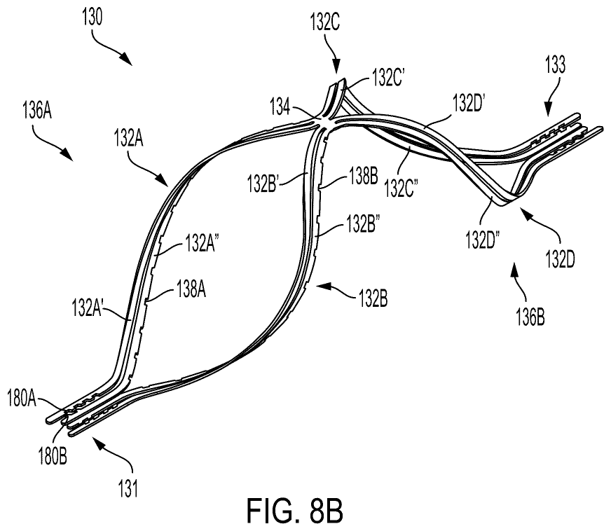

As shown in FIG. 8A, the arms 132A, 132B of the mesh support element 130 may include a plurality of notches. For instance, the arm 132A includes a plurality of notches 138A and the arm 132B includes a plurality of notches 138B. The notches 138A, 138B may be used for securing (e.g., directly coupling) the arms 132A, 132B to the mesh 140, as described further below. In some embodiments, the notches 138A, 138B may be omitted.

In some embodiments, one or more of the arms 132A, 132B, 132C, or 132D of the mesh support element 130 may include two or more arms rather than a single arm as illustrated in FIG. 8A. For example, FIG. 8B shows an embodiment of the mesh support element 130 in which the arm 132A includes two arms 132A′ and 132A″, the arm 132B includes two arms 132B′ and 132B″, the arm 132C includes two arms 132C′ and 132C″, and the arm 132D includes two arms 132D′ and 132D″. In such embodiments, when the mesh support element 130 includes the plurality of notches, the plurality of notches may be included on only the arms 132A″ and 132B″ as shown.

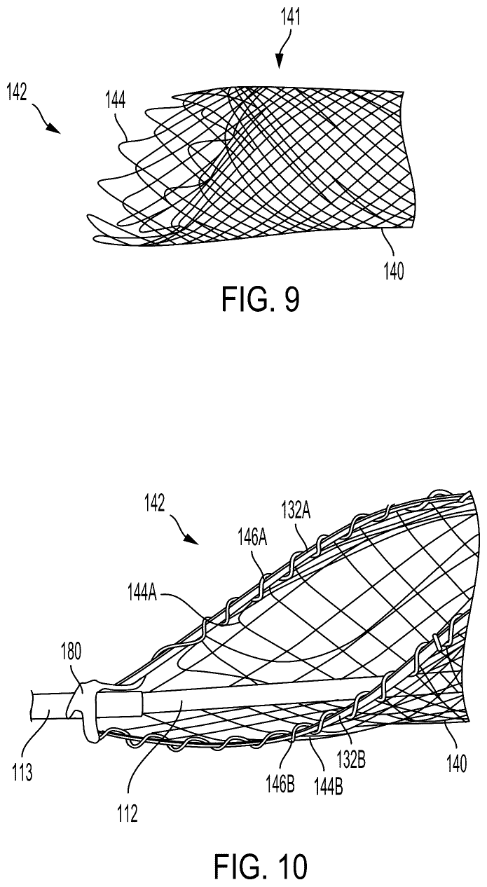

The connection of the mesh 140 to the coring element 120 will now be described. In some embodiments, the proximal end 141 of the mesh 140 may be secured (though not immovably) to the arms 132A, 132B via at least one wound wire, suture, or other suitable securing element. For example, FIG. 9 shows the proximal end 141 of the mesh 140 including a plurality of end loops 144. The plurality of end loops 144 may be secured to the arms 132A, 132B by at least one wire (e.g., a nitinol wire) wound around the arms 132A, 132B and through at least one or multiple of (e.g., each of) the plurality of end loops 144. For example, FIG. 10 shows a wire 146A wound around the arm 132A and through a first portion of the plurality of end loops 144, and a wire 146B wound around the arm 132B and through a second portion of the plurality of end loops 144. In some embodiments, the wires 146A, 146B may be a single wire that is wound through both the first and second portions of the plurality of end loops 144. Securing the proximal end 141 of the mesh 140 to the arms 132A, 132B in this way discourages the mesh 140 from shifting proximally during use.

In embodiments in which the arms 132A, 132B include the plurality of notches 138A, 138B, the wires 146A, 146B may be wound around the arms 132A, 132B such that at least a portion of the wires 146A, 146B is disposed within at least one of the plurality of notches 138A, 138B. Used in this way, the plurality of notches 138A, 138B discourage the wires 146A, 146B from sliding along the arms 132A, 132B. Used in this way, the plurality of notches 138A, 138B discourage the mesh 140 from shifting proximally during use. The at least one wound wire may further be attached to the mesh outer shaft 113 in any suitable fashion, including through the use of an adhesive. In other embodiments, one or more strands that are not wires (e.g., that are formed from suture material) may be used instead of the at least one wire to secure the proximal end of the mesh 140 to a proximal portion (e.g., arms 132A, 132B) of the mesh support element.

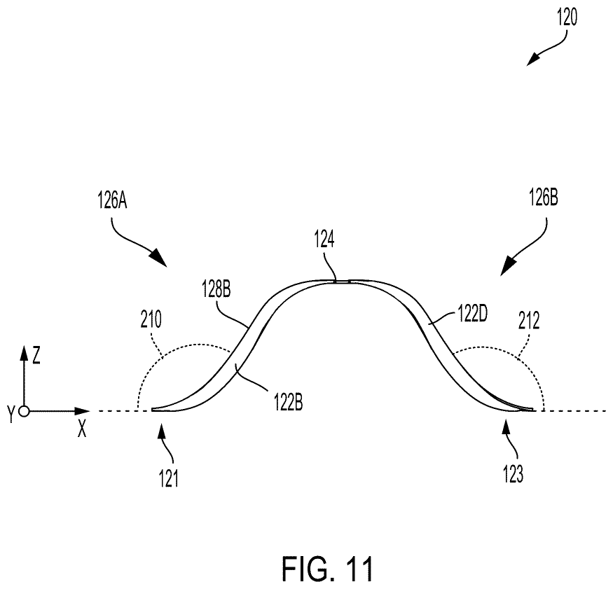

Each of the coring element 120 and the mesh support element 130 are shaped such that their respective arms are angled relative to the shaft assembly 110. The angled natures of those arms enables or otherwise helps the coring element 120 or the mesh support element 130 to collapse when either is pulled into an introducer sheath. Further, the angle of the coring element 120 can promote a sliding cutting action to cut through a stenotic lesion in an artery. As a representative example, FIG. 11 shows a side view of the coring element, though the description applies similarly to the mesh support element 130. In FIG. 11, a first plane including the x- and z-axes divides (e.g., bisects) the coring element 120 into a first side including the arms 122A and 122C, and a second side including the arms 122B and 122D. A second plane including the y- and z-axes (i.e. into and out of the page) divides (e.g., bisects) the coring element 120 into the first portion 126A and the second portion 126B. A third plane including the x- and y-axes (i.e. a plane perpendicular to the first and second planes) forms an angle 210 with the arms 122A, 122B and with the arms 122C, 122D.

While the angle 210 is shown to be measured relative to edges of the arms 122A, 122B (i.e., the tips of the blade portions 128A, 128B) in FIG. 11, the angle 210 may alternatively be measured from another portion of the arms 122A, 122B. The angle 210 is greater than 90° and less than 180°. For example, the angle 210 may be within one of the following ranges: 120 to 165°, 125 to 165°, 125 to 160°, 130 to 160°, 135 to 160°, 130 to 155°, 130 to 150°, 135 to 155°, or 135 to 150°. Similar to the arms 122A, 122B, the arms 122C, 122D may form an angle 212 with the third plane including the x- and y-axes. The description of the angle 210 applies similarly to the angle 212. In some embodiments, the angle 210 may be equal to the angle 212, though the angles 210, 212 are not limited in this way and may be unequal, including for different arms.

In the case of the mesh support element 130, with the proximal end 141 of the mesh 140 secured to the arms 132A, 132B, the mouth 142 of the mesh 140 also forms the angle 210 with the third plane including the x- and y-axes.

FIGS. 12A to 12C are a series of simplified depictions of the coring element 120 demonstrating the adjustability of the shape of the coring element 120. FIG. 12A depicts a first shape of the coring element 120 in which the arms 122A, 122B form the angle 210 with the third plane including the x- and y-axes. The coring element 120 also has a height H in the first shape. The height H is measured between the axis 220 and the bridge 124 of the coring element 120.

A position of the distal end 179 of the coring outer shaft 115 and a position of the distal end 175 of the coring inner shaft 114 may translate relative to one another along the axis 220. For example, the position of the distal end 179 of the coring outer shaft 115 is depicted as remaining stationary while a position of the distal end 175 of the coring inner shaft 114 translates in FIGS. 12A to 12C. In other examples, the coring inner shaft 114 may remain stationary while the coring outer shaft 115 translates, or both the coring inner shaft 114 and the coring outer shaft 115 may translate. The proximal end 121 and the distal end 123 of the coring element 120 may thereby translate relative to one another along the axis, which alters the shape of the coring element 120.

For example, the coring element 120 can transition into a second shape depicted in FIG. 12B. For example, when force applied to the bridge 124 toward the coring inner shaft 114 (e.g., by a vessel wall) increases as the diameter of the vessel decreases, the increase in force translates the distal end 175 of the coring inner shaft 114 away from the distal end 179 of the coring outer shaft 115 along the axis 220. In the second shape of the coring element 120, the angle 210′ is greater than the angle 210 and the height H′ is less than the height H.

Movement of the coring element 120 or of the coring inner shaft 114 or coring outer shaft 115 can further transition the coring element 120 into a third shape depicted in FIG. 12C. For example, as the vessel diameter decreases further, the force applied to the bridge 124 may increase further, which translates the distal end 175 of the coring inner shaft 114 farther away from the distal end 179 of the coring outer shaft 115 along the axis 220. In the third shape of the coring element 120, the angle 210″ is greater than the angle 210′ and the height H″ is less than the height H′. Each of the angles 210, 210′, 210″ may be within the ranges provided above for the angle 210. It will also be appreciated that, as the blade 128 is part of the arms 122A, 122B of the coring element, the blade 128 may be associated with any angle 210 and height H of the coring element 120 as well.

It will be appreciated that the mesh support element 130 may be adjusted similarly to the depictions of the coring element 120. For instance, the arms 132A, 132B of the mesh support element 130 form an angle similar to angle 210 with the third plane including the x- and y-axes. The mesh support element further includes a height similar to height H between the axis 220 and the bridge 124 of the coring element 120. Translating at least one of the mesh middle shaft 112 or the mesh outer shaft 113 relative to the other alters the height of the mesh support element 130.

It will also be appreciated that when the mouth 142 of the mesh 140 is coupled to an arm of each of the arms 132A, 132B of the mesh support element 130, the mouth 142 may be associated with any angle and height of the mesh support element 130 as well, meaning the values of those angles and/or those heights may be the same, or at least substantially the same, for the mouth (and therefore the open end) of the mesh as for the mesh support element. An advantage of the mesh support element 130 is that flexion and relaxation of the mesh support element 130 has little to no effect on the size (e.g., the area circumscribed by the mouth remains approximately the same) of the mouth 142 of the mesh 140. Rather, the mouth 142 remains approximately the same size despite the change in angle and height. In this way, a mouth 142 that is similarly sized for accepting clot into the mesh 140 can be deployed throughout any of the vasculature diameters from the IVC bifurcation to the common iliac vessel to the popliteal vessel.

Securement of each of the mesh support element 130 and the coring element 120 to the shaft assembly 110 will now be described. In some embodiments, the first portion 126A of the coring element 120 may be coupled to the coring outer shaft 115, and the second portion 126B to the coring inner shaft 114, via respective caps. For example, FIG. 13 shows an embodiment in which the distal end 123 of the coring element 120 is to be disposed within a cap 190. The cap 190 may include a metal or another suitable material. The cap 190 includes a gap 192 sized to accept the distal ends 123A, 123B of the arms 122C, 122D. For example, the distal ends 123A, 123B of the arms 122C, 122D may be positioned adjacent the distal end 175 of the coring inner shaft 114 and the cap 190 may be disposed over (e.g., forced over) the distal ends 123A, 123B, and 175. Alternatively, the cap 190 may be positioned adjacent the distal end 175 of the coring inner shaft 114 and the distal ends 123A, 123B of the arms 122C, 122D may be forced into the gap 192. An adhesive may join together the coring inner shaft 114, the arms 122C, 122D, and the cap 190.

In some embodiments, the cap 190 may be a polymer extrusion or a heat-shrink polymer sleeve positioned over the distal ends 123A, 123B and over a portion of the distal end of the coring inner shaft 114. In other embodiments, the cap 190 may be suture that is wrapped over the distal ends 123A, 123B and over a portion of the distal end of the coring inner shaft 114 to secure those distal ends together. Adhesive may be added to further strengthen the bond in such other embodiments.

The proximal end 121 of the coring element 120, including the proximal ends of the arms 122A, 122B, may be coupled to the coring outer shaft 115 in a similar manner as described above for coupling the distal ends 123A, 123B of the arms 122C, 122D to the coring inner shaft 114. For example, FIG. 14 shows the proximal end 121 coupled to the coring outer shaft 115 by a cap 190A, and the distal end 123 coupled to the coring inner shaft 114 by a cap 190B. In this example, the caps 190A and 190B each comprises suture that is wrapped over the distal ends of the depicted arms and over a portion of the distal end of the coring inner shaft 114 to secure those distal ends together, with adhesive added to further strengthen those bonds. In other embodiments, each of caps 190A and 190B may be a heat-shrink polymer sleeve.

Returning to FIG. 13, in embodiments in which the arm 122C or 122D includes two or more arms (e.g., arms 122C′ and 122C″ and arms 122D′ and 122D″), the distal ends of the two or more arms may each include engagement structures, such as respective opposing teeth. For example, in FIG. 8B, the arms 122A′ and 122A″ of the mesh support element 130 are shown having female teeth 180A and male teeth 180B, respectively. Similarly, the distal end of arm 122C″ may have male teeth 180B and the distal end of the arm 122C′ may have female teeth 180A. Interlocking of the male teeth 180B with the female teeth 180A fixes the positions of the arms 122A′ and 122A″ relative to one another. In some embodiments, one male tooth engaging one female tooth will suffice for the depicted purpose.

In some embodiments, instead of or in addition to the female teeth 180A and male teeth 180B, the distal ends of the arms 122A′ and 122A″ may be intertwined to coil together around the distal end 175 of the coring inner shaft 114. The cap 190 may similarly be positioned over the coiled arms 122A′ and 122A″, and adhesive similarly applied.

Each of the proximal end 131 and the distal end 133 of the mesh support element 130 may be secured to the shaft assembly 110 in any of the example manners provided above for the coring element 120. For example, the proximal end 131 of the mesh support element 130 may be secured to the mesh outer shaft 113 by a cap like cap 190 and the distal end 133 of the mesh support element 130 may be secured to the mesh middle shaft 112 by a cap like cap 190.

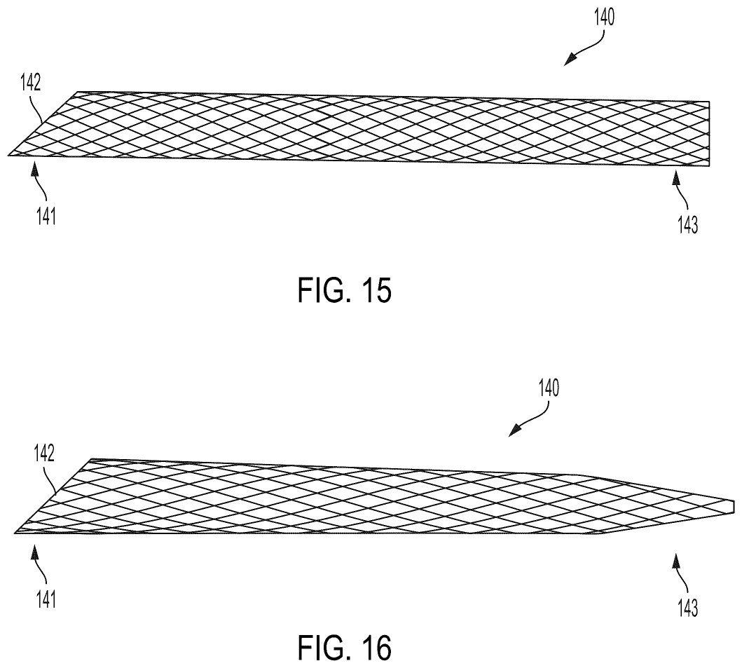

Example configurations of the mesh 140 will now be described. Referring to FIG. 15, in some embodiments, the mesh 140 may have a straight profile along a length of the mesh 140 such that a diameter of the mesh 140 adjacent to the mouth 142 is substantially equal to a diameter of the distal end 143 of the mesh 140.

Referring to FIG. 16, in some embodiments, the mesh 140 may have a tapered profile along the length of the mesh 140 such that a diameter of the mesh 140 adjacent to the mouth 142 is greater than a diameter of the distal end 143 of the mesh 140. In such embodiments, less material needs to be bunched up within the distal cap 150 compared to the mesh 140 having the straight profile. In an example, the mesh 140 having the tapered profile can be made, in part, by braiding the mesh 140 on a component machined into the shape of the tapered profile.

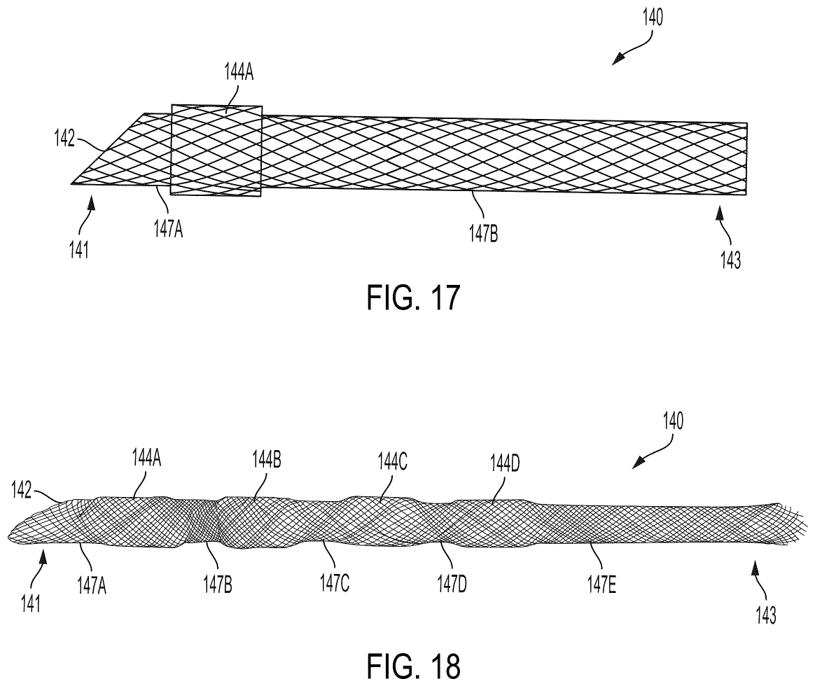

Referring to FIG. 17, in some embodiments, the mesh 140 may have a straight profile that includes a wider portion 144A (e.g., a bump). The bump 144A has a greater diameter than portions 147A, 147B (i.e., portions proximal and distal of bump 144A, respectively) of the mesh 140. In some aspects, the portions 147A and 147B have substantially equal diameters. The portion 147A includes the proximal end 141 of the mesh 140, and the portion 147B includes the distal end 143 of the mesh 140. In some embodiments, the portion 147B may have a tapered profile similar to the tapered profile of FIG. 16. The bump 144A can have a variety of widths or diameters in various aspects, and can also be disposed at various positions between the proximal end 141 and the distal end 143 of the mesh 140.

In some embodiments, the mesh 140 may include more than one bump 144A. For example, for a mesh 140 with a length within a range of 14-18 cm, the mesh 140 may include four bumps, which may be of similar size and shape. FIG. 18 shows an example of such a mesh 140 having a straight profile including four bumps 144A, 144B, 144C, and 144D. Portions 147A, 147B, 147C, 147D, and 147E of the mesh 140 have a smaller diameter than the bumps 144A-144D. In some aspects, the portions 147A-147E each have substantially equal diameters. The portion 147A includes the proximal end 141 of the mesh 140, and the portion 147E includes the distal end 143 of the mesh 140. In some embodiments, the portion 147E may have a tapered profile similar to the tapered profile of the embodiment of mesh 140 of FIG. 16.

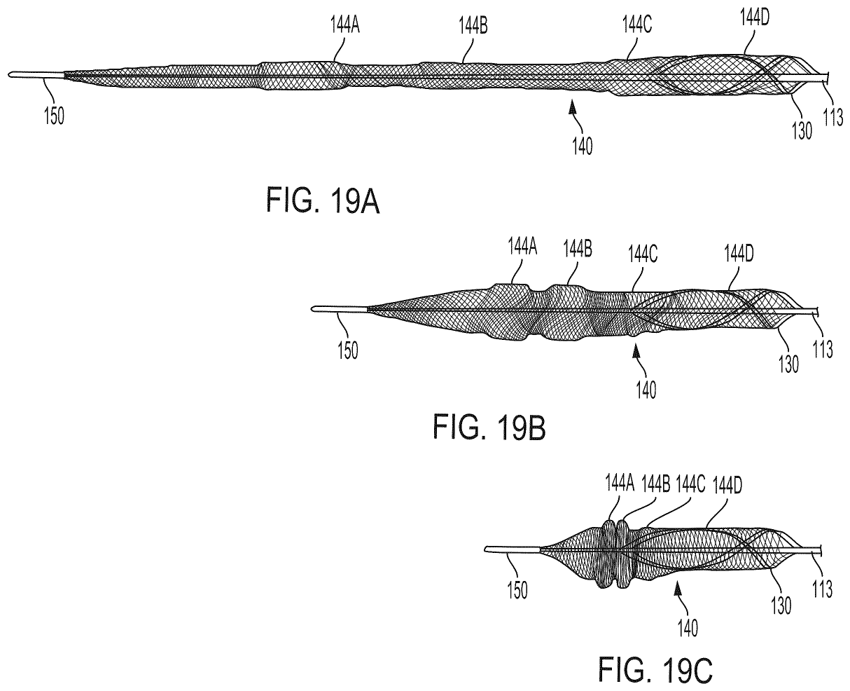

The one or more bumps 144A-144D enable the mesh 140 to compress axially without an outermost diameter (e.g., height) of the mesh 140 substantially increasing. For example, FIGS. 19A to 19C show three snapshots of the mesh 140 transitioning from an extended state to a compressed state. In FIG. 19A, the mesh 140 is shown in the extended state in which the mesh 140 is stretched and the outermost diameters of the bumps 144A, 144B, and 144C are less than the outermost diameter of the bump 144D. For example, to achieve the extended state, the mesh inner shaft 111 was advanced relative to the mesh middle shaft 112 and the mesh outer shaft 113. In FIG. 19B, the mesh 140 is shown in an intermediate state in which the outermost diameters of the bumps 144A and 144B are greater than the outermost diameters of the bumps 144C and 144D. For example, the mesh inner shaft 111 was retracted relative to the mesh middle shaft 112 and the mesh outer shaft 113 to transition the mesh 140 from the extended state to the intermediate state.

In FIG. 19C, the mesh 140 is shown in a compressed state in which the mesh is crunched into a compact shape and the outermost diameters of the bumps 144A, 144B, and 144C are marginally greater than the outermost diameter of the bump 144D. For example, the mesh inner shaft 111 was retracted further relative to the mesh middle shaft 112 and the mesh outer shaft 113 to transition the mesh 140 from the intermediate state to the compressed state. As shown in FIG. 19C, the outermost diameters of the bumps 144A and 144B did not substantially increase from the extended state to the compressed state while the length of the mesh 140 substantially decreased. The lack of substantial diameter increase is due, in part, to the material of the portions 147B-147D of the mesh 140 rolling within the bumps 144A-144D as the mesh 140 compresses.

Multiple advantages result from being able to compress the mesh 140 into a substantially shorter length without substantially increasing the outermost diameter of the mesh 140. For example, the compressed state of the mesh 140 enables advancing the clot extraction system 100 closer to an IVC filter to extract a greater amount of clot during a clot extraction procedure (e.g., prior to capturing clot material with the mesh 140). In another example, the mesh 140 must be cleaned of clot between passes of the extractor device 300 and it can be easier to clean the mesh 140 when the mesh 140 is in the crunched, compact shape of the (or another similar) compressed state. For instance, the distal end 143 of the mesh 140 can be reached more easily. The diameters and widths referenced with respect to the structures shown in FIGS. 15 to 19 are examples of transverse dimensions taken perpendicular to the axis of the extractor catheter (e.g., axis 220, shown elsewhere) or to any of the shafts.

Performance of the mesh 140 compressing into the crunched, compact shape can vary based on the shape and dimensions of the bumps 144A-144D, the portions 147B-147D, and the transitions between adjacent ones of bumps 144A-144D and portions 147B-147D. For instance, if the bumps 144A-144D are too pronounced (e.g., too great of height, too steep of transition angle, etc.) relative to the portions 147B-147D, it may become difficult for the material of one or more of the portions 147B-147D of the mesh 140 to roll up within one or more of the bumps 144A-144D as the mesh 140 compresses. In an example, a diameter of the bumps 144A-144D may be about 2 millimeters (mm) (e.g., 2 mm) greater than a diameter of the portions 147A-147E. In an example, each of the bumps 144A-144D may have a length within a range of 1 to 2 centimeters (cm). In another example, each of the portions 147B-147D may have a length within a range of 1 to 2 centimeters (cm). In some embodiments, a length of at least one of the bumps 144A-144D may be equal to a length of at least one of the portions 147B-147D. In some embodiments, a length of each of the bumps 144A-144D may be equal to a length of each of the portions 147B-147D. In some embodiments, a length of each of the portions 147B-147D may be shorter than a length of each of the bumps 144A-144D. In another example, there may be about a 2 millimeter (mm) (e.g., 2 mm) transition lengthwise between one or more of the bumps 144B-144D and a portion 147A-147E that is adjacent, which allows the diameter of the mesh 140 to gradually change.

The mesh 140 having the one or more bumps 144A-144D, in an example, can be made, in part, by braiding the mesh 140 on a component machined into the shape of the profile including the one or more bumps 144A-144D. In at least some embodiments, making the mesh 140 can further include heat treating the mesh 140 to set the shape.

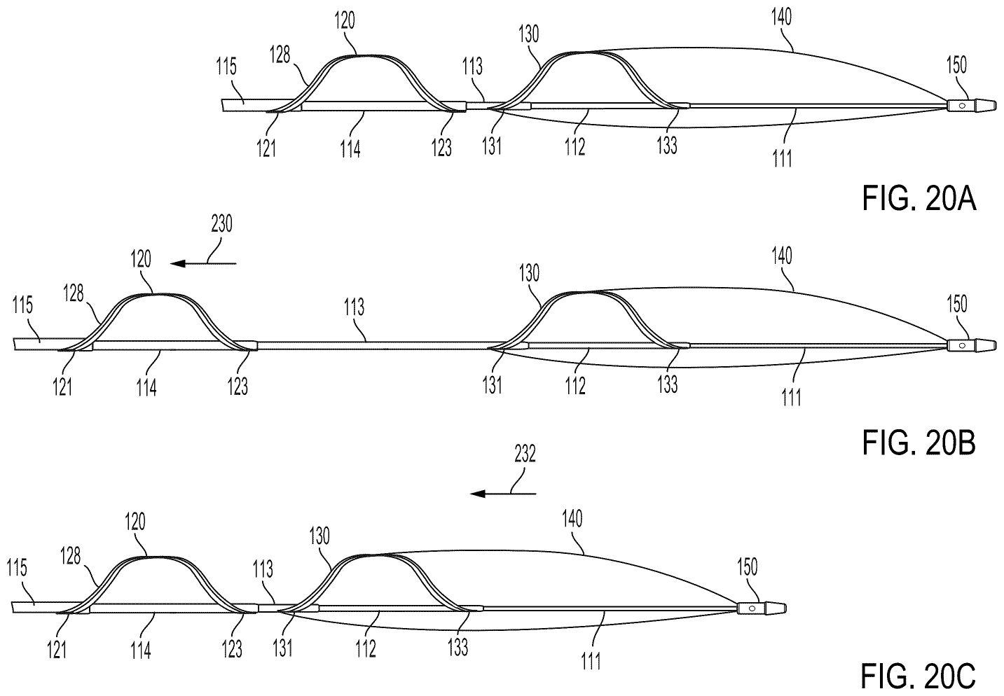

FIGS. 20A to 20C depict a method for extracting clot from a vascular system of a patient. Initially, the method includes deploying a clot extraction system (e.g., clot extraction system 100) within the vascular system of the patient. Deploying the clot extraction system 100 may include introducing the clot extraction system 100 into the vascular system through an introducer sheath (e.g., funnel sheath) and advancing the clot extraction system 100 to a desired position within the vascular system. The cover sleeve 116 may then be retracted to deploy the coring element 120, the mesh support element 130, and the mesh 140.

The clot extraction system 100 may, for example, be positioned as shown in FIG. 20A after initial deployment (e.g., over a guidewire). The method then includes retracting the coring element 120 (e.g., in the direction of arrow 230) a first amount (or length) through the vascular system while the mesh support element 130, and thereby the mesh 140, remains stationary. Stated differently, the coring element 120 is retracted relative to the mesh support element 130 and the mesh 140. At least a portion of a clot is removed from a wall of a vessel of the vascular system via the blade 128 by retracting the coring element 120. In some examples, the first amount is equal to a length of the coring element between the proximal end 121 and the distal end 123.

The mesh support element 130 and the mesh 140 are thereafter retracted (e.g., in the direction of arrow 232) a second amount through the vascular system while the coring element 120 remains stationary. Stated differently, the mesh support element 130 and the mesh 140 are retracted relative to the coring element 120. In some aspects, the second amount may equal, or approximately equal, the first amount. At least some of the portion of the clot that was removed from the wall of the vessel by the coring element is collected inside the mesh 140 by retracting the mesh support element 130 and the mesh 140. In some instances, the diameter of the blade 128 of the coring element 120 may then be reduced to a desired diameter, and the sequence repeated with the coring element 120 being retracted again, followed by retraction of the mesh support element 130. This sequence of may be repeated as many times as needed as the diameter of the vessel decreases.

Alternatively, the sequence may include setting a diameter of the blade 128 of the coring element 120, retracting the clot extraction system 100 as a whole for the first amount through the vascular system, reducing the diameter of the blade 128 to a desired diameter, and retracting the clot extraction system 100 as a whole another first amount. In this alternative sequence, the coring element 120 first breaks down clot during the first retraction of the clot extraction system 100, and then, after the diameter of the blade 128 is reduced, the coring element 120 breaks down additional clot while the mesh 140 collects the previously broken down clot during the second retraction of the clot extraction system 100.

In either embodiment of the sequence, the coring action can be independent from the collecting action when using the clot extraction system 100. Separating these two actions can have various benefits in the clot extraction process. For example, coring a first length of the vessel, followed by collecting the separated clot material along that first length of the vessel enables controlling a height (e.g., height H) of the blade 128 and a height (e.g., height H) of the mouth 142 of the mesh 140 based on a diameter of the vessel along the first length. The diameter of the vessel along the first length can be consistent, which reduces the force that the coring element 120 and the mesh support element 130 apply to the vessel wall since the coring element 120 and the mesh support element 130 are not forcing the vessel to expand along this first length. The heights of the blade 128 and the mouth 142, which correlate to diameters of the blade 128 and the mouth 142, can then be reduced before repeating the coring and subsequent collecting on a second length of the vessel, which has a smaller diameter than the first length.

In another example, the height of the blade 128 and the height of the mouth 142 of the mesh 140 can be controlled to different sizes when desired. For example, the coring action may be “turned off” when desired. For example, the coring element 120 may be retracted within the cover sleeve 116 when not needed, such as while the mesh 140, full of clot, is pulled through and out of the patient's vascular system, which eliminates the force of the blade 128 against the vessel wall when coring is not needed. In some embodiments, the coring element 120 may be retracted within the cover sleeve 116 after each amount the coring element 120 is retracted, while the mesh 140 collects the separated clot material, and can be deployed again once collecting is done to core the next amount of the vascular system. In another example, cutting the clot adhering to the vessel wall from the vessel wall in front of the mesh 140, which pushes the clot material toward the vessel's center, can make it easier to collect the clot material in the mesh 140 in some instances.

An alternative embodiment of the clot extraction system 100 will now be described. In the alternative embodiment, the clot extraction system 100 includes the cover sleeve 116 and a telescoping 3-shaft system that includes an inner shaft, a middle shaft, and an outer shaft. In one example of the alternative embodiment, the proximal end 121 of the coring element 120 is coupled to a distal end of the outer shaft, and the distal end 123 of the coring element 120 is coupled to a distal end of the middle shaft. Each of the proximal end 131 and the distal end 133 of the mesh support element 130 is coupled to the inner shaft. The proximal end 131 is fixed axially along the inner shaft whereas the distal end 133 is slidable along the inner shaft. In this way, the height of the mesh support element 130 relative to the inner shaft can adjust freely according to the resistance around the mesh support element 130 in a vessel. The proximal end 141 of the mesh 140 is coupled to the proximal end 131 of the mesh support element 130, and the distal end 143 of the mesh 140 is coupled to the distal end of the inner shaft.

In another example of the alternative embodiment, each of the proximal end 121 and the distal end 123 of the coring element 120 is coupled to the outer shaft. The proximal end 121 is fixed axially along the outer shaft whereas the distal end 123 is slidable along the outer shaft. In this way, the height of the coring element 120 relative to the outer shaft can adjust freely according to the resistance around the coring element 120 in a vessel. The proximal end 131 of the mesh support element 130 is coupled to the distal end of the outer shaft, and the distal end 133 of the mesh support element 130 is coupled to a distal end of the middle shaft. The proximal end 141 of the mesh 140 is coupled to the proximal end 131 of the mesh support element 130, and the distal end 143 of the mesh 140 is coupled to the distal end of the inner shaft.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

The invention claimed is:

1. A system comprising:
a first shaft;
a second shaft;
a third shaft;
a fourth shaft;
a fifth shaft;
a support element coupled to the second and third shafts;
a mesh having an open end and a second end, wherein the open end of the mesh is coupled to the support element and the second end of the mesh is coupled to the first shaft; and
a coring element coupled to the fourth and fifth shafts, wherein the coring element includes a cutting edge.

2. The system of claim 1, wherein no portion of the coring element is in direct contact with any portion of the mesh.

3. The system of claim 1, wherein a distal end of the coring element is positioned proximal of the open end of the mesh.

4. The system of claim 1, wherein a first portion of the coring element includes a first end that is coupled to the fifth shaft and a second portion of the coring element includes a second end that is coupled to the fourth shaft, and the first portion of the coring element includes the cutting edge.

5. The system of claim 4, wherein the coring element is configured such that an angle at which the cutting edge is disposed at an angle to the fifth shaft in an axial direction of the fifth shaft, and the angle is adjustable via movement of the fourth shaft relative to the fifth shaft in the axial direction.

6. The system of claim 1, wherein the first shaft is partially disposed within the second shaft, which is partially disposed within the third shaft, which is partially disposed within the fourth shaft, which is partially disposed within the fifth shaft.

7. The system of claim 1, wherein:
the open end of the mesh is disposed at an angle to an axial direction of the third shaft; and
the angle is within a range of 120 to 165 degrees.

8. The system of claim 1, wherein the support element includes a first end and a second end, the first end of the support element is coupled to the third shaft, and the second end of the support element is coupled to the second shaft.

9. The system of claim 1, wherein the support element is configured such that an angle at which the open end of the mesh is disposed relative to an axial direction of the third shaft is adjustable via movement of the second shaft relative to the third shaft in the axial direction.

10. A system comprising:
a telescoping shaft assembly including a plurality of shafts;
a coring element coupled to the telescoping shaft assembly, wherein the coring element includes a cutting edge;
a support element coupled to the telescoping shaft assembly and spaced apart from the coring element along the telescoping shaft assembly; and
a mesh coupled to the support element;
wherein the telescoping shaft assembly, the coring element, and the support element are configured such that the coring element is movable relative to the support element in a direction parallel to an axis extending through the telescoping shaft assembly.

11. The system of claim 10, wherein the coring element includes a first portion and a second portion, the first portion includes a first pair of arms, the second portion includes a second pair of arms, and each of the arms in the first pair of arms includes a portion of the cutting edge.

12. The system of claim 10, wherein the support element includes a first portion and a second portion, the first portion of the support element includes a first pair of arms, the second portion of the support element includes a second pair of arms, and each of the arms in the first pair of arms of the support element includes a plurality of notches.

13. The system of claim 12, wherein:
the mesh includes a plurality of end loops; and
the mesh is coupled to an arm of the first pair of arms of the support element by at least one wire wound around the plurality of end loops and the arm such that respective portions of the at least one wire are disposed within respective ones of the plurality of notches of the arm.

14. The system of claim 10, wherein a first portion of the coring element is secured to one shaft of the plurality of shafts, and a second portion of the coring element is secured to another shaft of the plurality of shafts.

15. The system of claim 10, wherein:
a segment of the coring element is disposed at an angle to an axis extending through the plurality of shafts; and
the angle is within a range of 120 to 165 degrees.

16. The system of claim 15, configured such that translation of a shaft of the plurality of shafts that is in direct contact with the coring element causes the angle of the segment to change.

17. The system of claim 10, wherein the cutting edge faces away from the support element.

18. An apparatus comprising:
a plurality of shafts;
a mesh comprising a distal end coupled to a first shaft of the plurality of shafts and an open end coupled to a second shaft of the plurality of shafts; and
a coring element comprising a first end coupled to a third shaft of the plurality of shafts and a second end coupled to a fourth shaft of the plurality of shafts, wherein the coring element includes a cutting edge;
wherein:
the open end of the mesh faces the coring element, and
the cutting edge of the coring element faces away from the mesh.

19. The apparatus of claim 18, further comprising a support element, wherein the open end of the mesh is coupled to the second shaft by the support element.

20. The apparatus of claim 18, configured such that the coring element is translatable in an axial direction independently from the mesh.

21. The apparatus of claim 18, wherein the mesh includes a first portion disposed between a second portion and a third portion, and the first portion of the mesh has a transverse dimension, taken perpendicular to an axis of one of the plurality of shafts, greater than a transverse dimension, taken perpendicular to the axis, of the second portion of the mesh and greater than a transverse dimension, taken perpendicular to the axis, of the third portion of the mesh.

22. The apparatus of claim 21, wherein the transverse dimension of the second portion of the mesh is greater than any other transverse dimension, taken perpendicular to the axis, of the second portion of the mesh, and the transverse dimension of the third portion of the mesh is greater than any other transverse dimension, taken perpendicular to the axis, of the third portion of the mesh.

23. The apparatus of claim 21, wherein the mesh is configured such that compressing the mesh by translating the distal end of the mesh toward the open end of the mesh causes the third portion of the mesh to roll up within the first portion of the mesh.
